(19) United States
(12) Patent Application Publication
(10) Pub. No.: US 2004/0185120 A1
(43) Pub. Date: Sep. 23, 2004

(54) Wound Flush

(75) Inventor: Linda K. Grant, Kewanee, IL (US)

Correspondence Address: LINDA K. GRANT, 10128 E. 2300 ST., KEWANEE, IL, 61443, US

(73) Assignee: Ms. Linda Kay Grant, Kewanee, IL (US)

(21) Appl. No.: 10/249,203

(22) Filed: Mar. 21, 2003

(51) Int. Cl. A61K 35/78

(52) U.S. Cl. 424/725

(57) ABSTRACT

Not to be taken internally.

### DETAILED DESCRIPTION

A natural wound wash with safe natural ingredients that sterilizes the wound area and hastens the healing process. Also, promotes hair growth and tissue regeneration to minimize scarring.

Contains sterile water, iodine, and softening agents in appropriate amounts.

1. When used to flush fresh wounds, it immediately cleanses and sterilizes the area.

2. Starts healing process and promotes rapid growth of new hair and tissue. When used to flush fresh wounds, it immediately cleanses and reduces scarring.

3. Reduces scarring
